(19) United States
(12) Patent Application Publication
(10) Pub. No.: US 2007/0276485 A1
(43) Pub. Date: Nov. 29, 2007

(54) Implant With Improved Homogeneity for Plastic Surgery and Method for the Production Thereof

(76) Inventor: Pascal Paganon, St Symphorien D'Ozon (FR)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 10/565,613

(22) PCT Filed: Jul. 16, 2004
(86) PCT No.: PCT/FR04/01890

§ 371 Date: Feb. 21, 2007

(30) Foreign Application Priority Data
Jul. 24, 2003 (FR) 0309102

(51) Int. Cl. A61F 2/02

(52) U.S. Cl. 623/8; 623/11.11

(57) ABSTRACT

An implant and a method for producing an implant for plastic surgery comprising an injection stage, wherein an elastomeric material is injected into a mould in order to produce a flexible envelope for forming a pocket.

### PRIORITY CLAIM

This patent application is the U.S. National Phase of International Application No. PCT/FR/2004/001890, having an International Filing Date of Jul. 16, 2004, which claims priority to French Patent Application No. FR-03 09102, having a Filing Date of Jul. 24, 2003, the disclosures of which are incorporated herein by reference in their entirety.

### FIELD OF THE INVENTION

This invention relates to the technical field of artificial devices of the implant or prosthesis types, designed to remodel the shape of part of the human body, for aesthetic and/or therapeutic purposes.

This invention relates more particularly but not exclusively to the domain of breast implants designed to assure breast reconstruction following a mastectomy, or to increase the size of the breasts for aesthetic purposes.

This invention relates to a plastic surgery implant designed to be implanted in the body of the patient, the said implant comprising at least one flexible pouch delimiting a predetermined internal volume, the said flexible pouch being formed from at least one envelope made from an elastomer material.

This invention also relates to a process for making a plastic surgery implant.

### BACKGROUND OF THE INVENTION

It is known that a foreign body can be implanted in the body of the patient to perform plastic surgery, either to reconstruct a part of the body for example following an accident or a disease, or for aesthetic purposes to modify the appearance of part of the body.

In particular, it is known that an implant can be placed in a patient's breast to create a breast implant in order to reconstruct and increase the volume of the breast following a mastectomy and/or for aesthetic purposes.

It is also known that such foreign bodies can be implanted for purely aesthetic purposes in the buttocks, the legs, the arms or for example the pectoral muscles.

These foreign bodies are generally referred to as a “prosthesis” or “implant”, and are usually in the form of a flexible envelope, for example made from a biocompatible elastomer material, containing a quantity of a filling material, for example such as a silicone gel or physiological serum which gives the functional volume to the prosthesis.

These implants are usually inserted subcutaneously by making an incision close to the final implantation zone of the implant.

In the special case of breast implants, they are usually inserted subcutaneously at the patient's thorax, on or under the pectoral muscle, by making an incision that may be made close to the armpit, along the sub-mammary groove, or around the areola of the nipple.

Such plastic surgery implants are usually made by dipping.

The dipping process consists of dipping a core with the required shape for the implant (for example hemispherical, ovoid, ellipsoid) in a silicone bath dispersed in a solvent, drying the film formed on the surface of the core by this dipping, and then “stripping” the core to obtain a flexible pouch.

Another frequent technique is insert moulding of the pouch obtained by dipping so as to obtain a flexible envelope formed from a stack of layers.

Each layer may be different from the others, the surface layer in particular having texture properties facilitating its implantation.

Such a dipping manufacturing process is generally satisfactory, but it has many disadvantages.

Due to the presence of an inflammable and toxic solvent in the dipping bath, special equipment has to be used both for the machines and for the civil works (explosion-proof room) to guarantee the safety and health of operators.

Furthermore, this process is particularly difficult to implement because it requires precise control over the fluidity of the dipping bath, which requires surveillance and constant re-supply of solvent, since the solvent is usually very volatile. Therefore, this process requires highly qualified personnel.

This process also includes a large number of tasks particularly justifying the use of a conveying carousel, such that the resulting cycle time to obtain an implant is relatively long.

Moreover, the dipping process is generally incapable of precisely controlling the thickness of the envelope of the implant. Implants obtained by dipping are generally satisfactory, but they do not have sufficient dimensional precision resulting in overthickesses in some parts of the implant which increases production costs of the implant, or underthicknesses in other places which could possibly cause weakening of the implant.

Finally, the scope of this process is limited particularly because it cannot be used to achieve controlled variations of the thickness and/or shape on the same part. Consequently, the design of a plastic surgery implant to be produced using this process depends on a liquid running on a core, which considerably reduces the possibilities of combining shapes, dimensions and finally functions, on the same part.

### SUMMARY OF THE INVENTION

The features of the present invention are to correct the various disadvantages listed above and to propose a new plastic surgery implant with higher strength and better uniformity.

Another feature of the invention is to propose a new process for fabricating a plastic surgery implant to be used for simpler and more reliable fabrication of implants, at lower cost.

Another feature of the invention is to propose a new process for fabricating a plastic surgery implant to fabricate implants quickly and with an excellent dimensional uniformity.

Another feature of the invention is to propose a new process for fabricating a plastic surgery implant, with a smaller number of steps.

The features of the invention are achieved using a process for fabrication of a plastic surgery implant characterised in that it comprises an injection step in which an injection press is used to inject an elastomer material into a mould to obtain a flexible envelope that will participate in the formation of a pouch (2).

The features of the invention are also achieved by means of a plastic surgery implant to be implanted into the body of a patient, the said implant being obtainable by a fabrication process according to one of claims 1 to 5, characterised in that the said implant comprises at least one flexible pouch delimiting a predetermined internal volume, the said flexible pouch being formed from at least one envelope made from an elastomer material, the dimensional tolerance of the thickness of the said at least one envelope being between 1% and 20%.

### BRIEF DESCRIPTION OF THE DRAWINGS

Other features and advantages of the invention will become clearer after reading the following description with reference to the appended drawings that are given purely for illustrative and non-limitative features, in which:

FIG. 1 shows a side view of a mould in a closed position used to implement the process according to the invention,

FIG. 2 shows a side view of the mould in FIG. 1 in the open position, and in the stripping position,

FIG. 3 shows a diagrammatic view of a plastic surgery implant with a multi-layer pouch according to the invention,

FIGS. 4 and 5 diagrammatically show a top view and a side view respectively of a first exemplary embodiment of a breast implant according to the invention,

FIGS. 6 and 7 diagrammatically show a top view and a side view respectively of a second exemplary embodiment of a breast implant according to the invention.

FIGS. 8 and 9 diagrammatically show a top view and a side view respectively of a third exemplary embodiment of a breast implant according to the invention.

### DESCRIPTION OF THE INVENTION

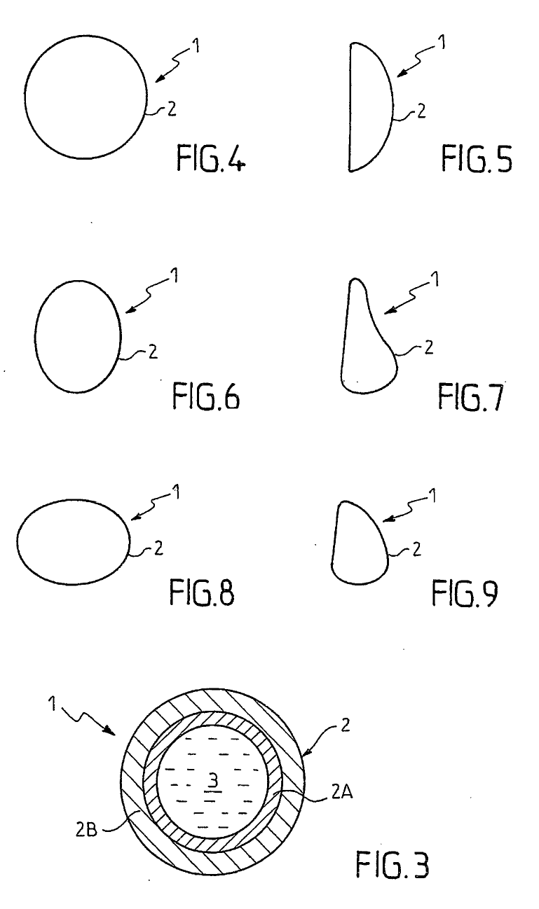

FIGS. 3 to 9 show a plastic surgery implant 1 according to the invention.

In the following, “plastic surgery” means surgery designed to modify the shape of an organ or a part of the body, so as to correct a congenital or acquired anomaly, and/or to modify the aesthetics of the body of a patient, for example to increase the volume.

The plastic surgery particularly includes a branch called “aesthetic surgery” that is particularly applicable to remodelling of parts of the body for essentially aesthetic purposes.

Therefore, this invention relates to plastic surgery, and preferably aesthetic surgery.

The plastic surgery implant 1 conforming with the invention is designed to be inserted under the skin of a patient to reconstruct and/or remodel and/or increase the volume of a part of the body, for example a breast, to the extent that the said implant that has a predetermined functional volume increases the size of or replaces biological tissues, for example and preferably breast tissues.

The insertion of the plastic surgery implant 1 conforming with the invention is usually and preferably done by inserting the said implant 1 subcutaneously by making an incision previously made by the surgeon.

In the special case in which the implant 1 is a breast implant (see FIGS. 4 to 9), the said breast implant may in particular be implanted using an axillary, sub-mammary, peri-areolar or trans-areolar method.

The plastic surgery implant according to the invention may for example be an arm implant (forearm, biceps), a leg implant (calves, thighs), a buttock or a chest implant (breast implant for women, pectoral implant for men). The breast implant will be the preferred example of plastic surgery implants within the scope of the invention.

The plastic surgery implant 1 according to the invention conventionally comprises a flexible pouch 2 delimiting a predetermined internal volume, controlled by its external walls.

The said at least one flexible pouch 2 is preferably made from elastomer materials, for example based on one or several silicones. The flexible pouch 2 may be made using a single layer construction, in other words it is formed from a single envelope (not shown) or a multilayer method, in which the flexible pouch 2 is formed from the stack of different envelopes 2A, 2B possibly with different compositions and/or properties.

In particular, the flexible pouch 2 may be a complex formed from a stack of layers 2A, 2B fixed relative to each other, preferably over their entire surface of each layer. Therefore, the flexible pouch 2 is preferably a single-piece unit.

Such a multilayer construction can provide a properties gradient depending on the thickness of the pouch 2, in other words for example an excellent seal provided by the internal layer 2A, and a “velvety” texture conferred by the external layer 2B.

Without going outside the scope of the invention, it would also be possible for the implant 1 to include two pouches (not shown) with different volumes, one contained in the other and each containing a different filling material.

The following description refers particularly to a single flexible pouch 2 formed from a two-layer complex and made by stacking an internal layer 2A and a surface layer 2B, as shown in FIG. 3.

However, without going outside the scope of the invention, it would be possible for the flexible pouch 2 to comprise a larger number of layers, for example three, four, five or more.

Conventionally, the predetermined internal volume defined by the flexible pouch 2 contains a filling material 3, which gives the required consistency and functional volume to the implant 1.

As a person skilled in the art is well aware, the filling material 3 may be a liquid, a gel or even a gas. For example, the filling material 3 could be a silicone gel, a saline solution, a physiological liquid, soya oil, polyvinylpyrrolidone (PVP) or a hydrogel.

According to one important characteristic of the invention, the dimensional tolerance T of the nominal thickness eₙₒₘ of at least one of the envelopes 2A, 2B participating in formation of the pouch 2, is between 1% and 20%, in other words the real thickness eᵣₑₐₗ of the envelope 2A may vary between eₙₒₘ(1−T) and enom(1+T).

In other words, the general concept of the invention is based on the presence of an envelope 2A, 2B with a tolerance T between 1% and 20%, regardless of whether this envelope alone forms a pouch 2 or if it is associated with other envelopes in a multilayer structure.

In the case of a multilayer structure (for example shown in FIG. 3), one or several or even all envelopes 2A, 2B may have the dimensional tolerance characteristic conforming with the invention.

For example, if the dimensional tolerance T is 10%, namely 0.1, the real thickness eᵣₑₐₗ may vary between eₙₒₘ(1−0.1) and eₙₒₘ(1+0.1), in other words between 0.9 eₙₒₘ and 1.1 eₙₒₘ, for the envelope 2A, 2B considered.

Any metrological method usually used in industry can be used to check if a particular envelope 2A, 2B actually satisfies the dimensional tolerance mentioned above.

For illustrative and non-limitative purposes only, a metrological method could be adopted using the following steps:

a. calculate the deviation E using the following formula:
E=100(eₘₐₓ−eₙₒₘ)/eₙₒₘ if |eₘₐₓ−eₙₒₘ|≧|eₘᵢₙ−eₙₒₘ|

or
E=100(eₙₒₘ−eₘᵢₙ)/eₙₒₘ if |eₘₐₓ−eₙₒₘ|≦|eₘᵢₙ−eₙₒₘ|

where: eₙₒₘ could be considered to be the arithmetic mean of envelope thicknesses recorded at a significant number N of measurement points distributed on the envelope, eₘᵢₙ being the minimum measured thickness on the N points and eₘₐₓ being the maximum measured thickness on the N points.

a. comparison of E with T:

if E|≦|T, the checked envelope is conforming with the purpose of the invention.

The thickness measurement may be made using a mechanical thickness comparator, for example the Mitutoya mechanical comparator NO7304.

Obviously, all N measurement points must be chosen such that they are all applicable to the same material category. Thus, care will be taken in general to assure that no measurement point is placed on a singularity of the envelope 2A, 2B, regardless of whether this singularity consists of a joint plane, a reinforcement (for example at a valve), or any other element.

Advantageously, the dimensional tolerance of the thickness of envelope 2A, 2B is within a range varying from 15% to 20%. For example, it is worth mentioning the case of a silicone envelope with a nominal thickness equal to approximately 0.5 mm with a tolerance between 15% and 20%. This means that the nominal thickness of the envelope 2A, 2B is 0.5 mm, while the real thickness can vary from between 0.5±0.075 mm (when the tolerance T is equal to 15%) to between 0.5±0.1 mm (when the tolerance T is equal to 20%).

In the above, we considered the case of an envelope 2A, 2B made of silicone, or based on silicone. However, it would be quite possible for this envelope to be made from any other material, and particularly elastomer, without going outside the scope of the invention.

The invention also relates to a process for fabrication of a plastic surgery implant 1, the said implant being designed to be implanted subcutaneously into the body of a patient.

Preferably, the invention relates to a process for fabrication of a plastic surgery implant from the following group:
breast implant,
pectoral implant,
leg implant,
arm implant,
buttocks implant.

According to one important characteristic of the invention, the fabrication process comprises an injection step in which an injection press is used to inject an elastomer material such as silicone into a mould to obtain a flexible envelope 2A, 2B that will participate in the formation of the pouch 2 of the implant 1.

Therefore, the fabrication process is an injection moulding process performed using an injection press, in other words an injection moulding press. Therefore, the term “injection” in this description must be understood as being related to an operation performed using a press, rather than a simple transfer of material such as by pouring, which could be done without an injection press.

Preferably, the injected elastomer material is not very foaming or foamy, and is more in “solid” form.

In general, the process according to the invention is designed to eliminate practically all air bubbles that could be trapped within the elastomer material. Thus, the process can be used to obtain a regular and homogenous flexible envelope that can be used in particular to form the outside shell of a surgical implant.

Advantageously, the mould comprises:

an upper cavity 40 comprising a concave conformation 40A forming a hollow defining a portion of the surface of the flexible envelope to be obtained,

a lower cavity 41, comprising a concave conformation 41B that defines a hollow defining a surface complementary to the surface of the upper cavity 40, such that when the upper cavity 40 comes into contact with the complementary lower cavity 41, the result is a closed internal volume with a fairly good seal delimited by a surface 40A, 41A, with a shape that corresponds approximately to the shape of the required flexible envelope 2A, 2B.

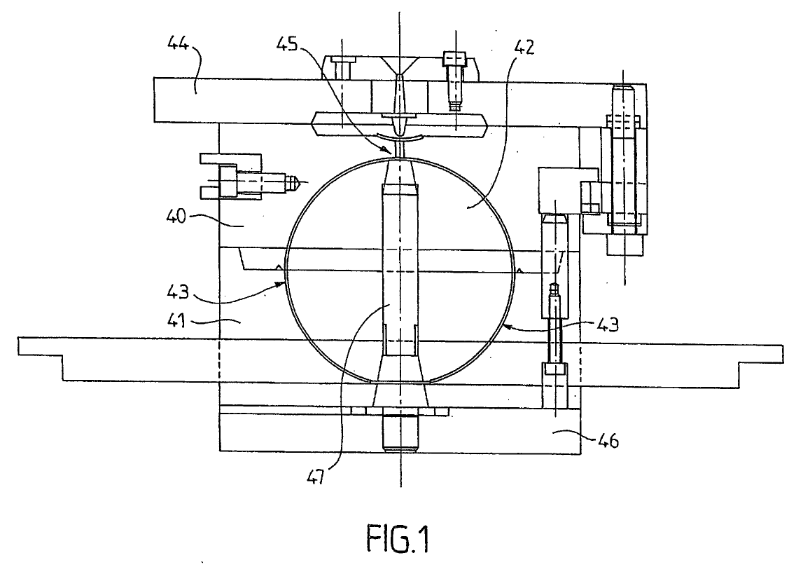

The mould also comprises a core 42 formed by a convex body for which the outside surface is practically complementary to the outside surface of the internal volume defined by the upper cavity 40 and the lower cavity 41, except on a different scale. The outside surface of the core 42 is thus geometrically similar to the surface of the internal volume defined by the upper cavity 40 and the lower cavity 41, but is smaller. The core 42 will be positioned within the internal volume, preferably equidistant from the walls defining the internal volume. The result is thus described in FIG. 1, in which the upper cavity 40 and the lower cavity 41 surround the core 42, so as to define an interstice or air gap 43 that is a free space delimited firstly by the outside surface 42A of the core 42, and secondly by the inside surface 40A, 41A of the internal volume defined by the upper cavity 40 associated with the complementary lower cavity 41.

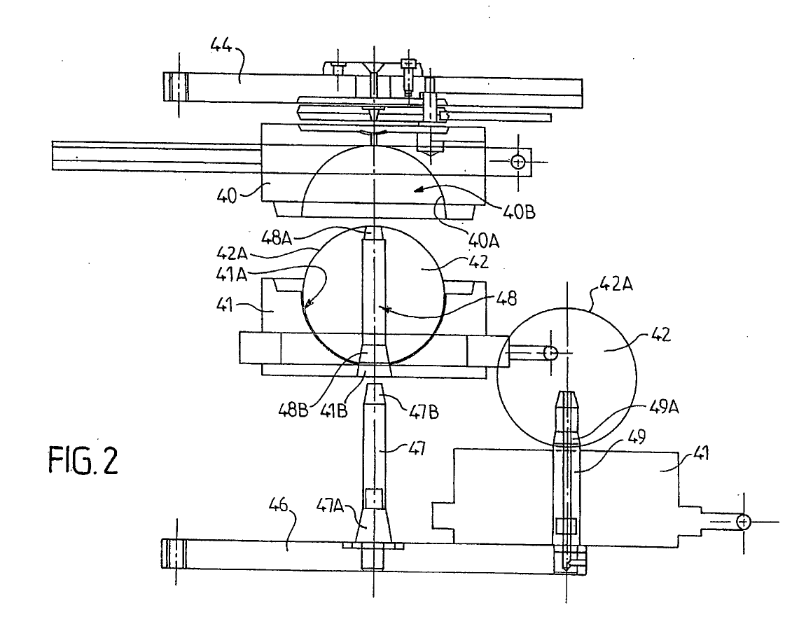

In the purely illustrative case corresponding to FIGS. 1 and 2 in which it is required to obtain an approximately spherical and single-piece envelope, the injection process thus includes a mould preparation step before the injection step, in which a generally hemispherical-shaped upper cavity 40 is brought into contact with a complementary generally hemispherical-shaped lower cavity 41, so as to obtain an approximately spherical internal volume, after positioning the two cavities 40, 41 concentrically with the said internal volume, a spherical core 42 with a diameter less than the diameter of the said internal volume defined by the upper cavity 40 and the lower cavity 41. This mould preparation step is followed by an injection step in which an elastomer material such as for example gum silicone or liquid silicone is injected into the interstitial space between the core 42 and the upper cavity 40 and the lower cavity 41, so as to obtain a generally spherical shaped envelope 2A, 2B that will participate in forming the pouch 2 of the implant.

Advantageously, the cavity 40 is fixed to an upper flange 44, such that the inner space 40B defined by the concave conformation 40A of the cavity 40 is in fluid communication with the upper flange 44, that itself supports the elastomer material injection means, that are themselves in communication with the injection press (not shown).

The injection means preferably include three injection nozzles distributed at regularly distributed angles (spacing of 120°) around or at the summit 45 of the internal volume defined by the upper cavity 40 and the lower cavity 41. The summit 45 is thus approximately at the concave conformation point 40A of the upper cavity 40 closest to the upper flange 44.

Preferably, all injection nozzles have exactly the same flow.

Advantageously, the process also uses a lower flange 46 on which a centring shaft 47 of the core 42 is fixed. The core 42, which may for example be in the form of a solid sphere as shown in FIGS. 11 and 12, comprises a reaming 48 with a shape complementary to the shape of the centring shaft 47 so that the core 42 can be force fitted onto the shaft 47, through the lower cavity 41 in which a slot 41B is formed for the centring shaft 47 to pass through.

The reaming 48 and the centring shaft 47 are designed to limit any risk of seizure between the core 42 and the centring shaft 47. Consequently, the steels from which the core 42 and the centring shaft 47 are made must preferably have different hardnesses, for example 49 or 50 HRc for the centring shaft 47, and 35 HRc for the core 42 (Rockwell hardness).

It is also envisaged to be able to provide the centring shaft 47 with a generally tapered base 47A, and an also generally tapered opposite engagement end 47B. The reaming 48 formed in the core 42 comprises a lower recess 48B at each of its ends with a shape complementary to the shape of the base 47A, and an upper recess 48B with a shape complementary to the shape of the engagement end 47B, the said centring shaft 47 and the reaming 48 being arranged so as to facilitate annular bearing between the base 47A of the centring shaft 47 and the core 42, the said annular bearing contributing to control over positioning of the core 42 with respect to the centring shaft 47.

The stripping operation is performed using a stripping shaft 49, one of the ends is fixed to the lower flange 46, while the other end or the leading end 49A is designed to engage the lower recess 48B of the core 42, and consequently is provided with a conformation authorising the lower end 48B of the reaming 48 to stop in contact with it. The core 42 of the lower cavity 41 can thus be extracted as follows:

the upper cavity 40 and the lower cavity 41 are separated,

the lower cavity 41 is then moved in a vertical translation movement along the direction of the centring shaft 47, so as to take the core 42 away from the centring shaft 47,

the sub-assembly formed by the lower cavity 41 supporting the core 42 is then inserted on the stripping shaft 48 so as to extract the core 42 outside the concave conformation 41 A of the lower cavity 41.

The final step is actual stripping of the silicone envelope matching the surface of the core 42. The core 42 will advantageously be covered by a surface treatment coating limiting adhesion of elastomers, for example a uniform layer of Teflon® a few micrometers thick, to facilitate this operation.

Thus, the process according to the invention provides a means of quickly producing a flexible envelope with a very uniform wall thickness, using a single machine and in a limited number of operations (the cycle time may be of the order of 5 minutes). For example, an envelope 2A, 2B made of silicone with a nominal thickness equal to 0.5 mm can be made with a tolerance of less than ±0.08 mm and possibly ±0.05 mm.

Advantageously, the process according to the invention includes a complexing step after the reinjection step in which the flexible envelope 2A obtained after the injection step is covered with a surface envelope 2B by a dipping operation, to obtain a flexible pouch 2 similar to that shown in FIG. 3.

This dipping operation preferably takes place as follows:

the flexible envelope 2A obtained by injection is inserted on a dipping core,

the assembly formed by the core covered with the flexible envelope 2A is dipped in a suitable bath using any well-known dipping technique, so as to cover the flexible envelope 2A with a surface envelope 2B, for example made of silicone.

This provides a means of combining the benefit of fast injection with texture effects such as a “velvet” type texture, that can be obtained by dipping.

The result is thus a flexible pouch 2 in the form of a multilayer complex, in other words a material formed by stacking of different material layers.

There are two of these layers in the example in FIG. 3, it being understood that a larger number of layers will be quite possible without going outside the scope of the invention.

In the case of a multilayer pouch 2, it would also be possible for each of the envelopes 2A, 2B forming the pouch 2 to be obtained by injection, without going outside the scope of the invention.

Many shapes of implants can be made using the process according to the invention, and particularly breast implant shapes like those shown diagrammatically in FIGS. 3 to 9.

The invention can be used in industrial applications for the fabrication of plastic surgery implants.

1. A process for fabrication of a plastic surgery implant, injecting an elastomer material using an injection press into a mould to obtain a flexible envelope that will participate in the formation of a pouch.

2. The process of claim 1, further comprising a mould preparation step before the injection step, comprising contacting an upper cavity with a lower cavity with a complementary shape so as to obtain a closed internal volume, after positioning a core with a volume smaller than the said internal volume, between the two cavities, the injection step including injection of an elastomer material into the interstitial space between the core and the cavities.

3. The process of claims 1, further comprising a complexing step after the injection step comprising covering said flexible envelope with a surface envelope.

4. The process of claims 1, wherein the injected elastomer material is substantially non-foaming.

5. Process The process of claim 1, wherein said implant is selected from the group consisting of:
breast implant,
pectoral implant,
leg implant,
arm implant, and
buttocks implant,
of claims 1.

6. A plastic surgery implant produced by the process of of claims 1, wherein said implant comprises at least one flexible pouch delimiting a predetermined internal volume, said flexible pouch being formed from at least one envelope made from an elastomer material, the thickness dimensional tolerance of the said at least one envelope being between about 1% and about 20%.

7. The plastic surgery implant of claim 6, wherein the tolerance is within a range between about 15% and about 20%.

8. The plastic surgery implant of claim 6, wherein the thickness of said at least one envelope is about 0.5 mm with a tolerance between about 15% and about 20%.

9. The plastic surgery implant of claim 6, wherein said at least one envelope is made from silicone or a silicone-based material.

10. The plastic surgery implant of claim 6, wherein said implant is selected from the group consisting of:
breast implant,
pectoral implant,
leg implant,
arm implant, and
buttocks implant.

11. The process of claim 2, further comprising a complexing step after the injection step in which the said flexible envelope is covered with a surface envelope by a dipping operation.

12. The plastic surgery implant of claim 7, wherein the thickness of said at least one envelope is about 0.5 mm with a tolerance between about 15% and about 20%.

13. The process of claim 3, wherein said coating step is achieved by dipping said flexible envelope.
